(19) United States
(12) Patent Application Publication
(10) Pub. No.: US 2019/0309246 A1
(43) Pub. Date: Oct. 10, 2019

(54) System And Method For Cultivating Biological Organisms

(71) Applicant: JGM Industries Pty Limited, Hornsby NSW (AU)

(72) Inventor: Colin James McGregor, Hornsby (AU)

(21) Appl. No.: 16/346,776

(22) PCT Filed: Nov. 1, 2017
(86) PCT No.: PCT/AU2017/051202

§ 371 Date: May 1, 2019

(30) Foreign Application Priority Data
Nov. 1, 2016 (AU) 2016904455

(51) Int. Cl. C12M 1/00; A01G 33/00; A01K 63/04; A01K 61/20; C12M 1/26; C12N 1/12

(52) U.S. Cl. CPC C12M 23/18; A01G 33/00; A01K 63/047; A01K 61/20; C12N 1/12; C12M 33/14; C12M 21/02; C12M 29/18; A01K 63/045

(57) ABSTRACT

A flow passage (70) is shown in the form of two successively linked, substantially parallel, adjacent, open top channels (12A, 14A) to form part of an algae cultivation system. The forward flow of the water passes in sequence through the respective open top channels (12A) and (14A) of flow passage (70), in the direction of arrow ‘A’ and then is then recirculated back into to the channel (12A), and so on. The combination of added nutrients, sunlight and agitation of the fluid flow encourages the growth cultivation of the biological organisms which are continually suspended in the flowing water. Such a flow passage (70) can be used as a standalone passage used as part of a system for cultivation of biological organisms, or it can also form one basic unit of a modular system which is arranged in use to have several such flow passages linked together in use. Such a reconfigurable apparatus gives the user the flexibility to cultivate different algae products both separately and simultaneously.

### TECHNICAL FIELD

This disclosure relates generally to a system and method for growing one or more biological organisms and, in particular a method for optimising the growth conditions for photosynthetic algae, which can be suitable for use as a food source or as a dietary supplement in humans. Algae can also find use as a stock feed or a stock feed ingredient, for farm animals such as chickens, cattle, and pigs, as well as being a food source for all forms of aquaculture.

While the present disclosure is mainly concerned with a system and method for this purpose, it can also be applied to growth and development of other types of biological organisms, biomass and biomass-derived products such as biofuels, bacteria and yeasts, vitamins and minerals in various forms, and basic sea creature food sources such as spawn and plankton.

### BACKGROUND TO THE DISCLOSURE

Algal species inhabit most marine environments, such as lakes, ponds, rivers and reservoirs. On occasion, the algae can experience a “bloom” or rapid period of growth which dominates the other flora and fauna present in the waterway. Algae such as blue-green algae is a cyanobacterium which can have both beneficial and detrimental properties to life. For example, Spirulina is a dietary food supplement which is a form of cyanobacterium which is safe to consume as a replacement for protein in humans, farm animals and in aquaculture. Dried Spirulina may contain anywhere from 50-70% protein, depending on its source, and Spirulina is also a supply source of B-vitamins and dietary minerals, such as iron and manganese.

In the growth of an algae to produce Spirulina, some cyanobacterium are also known to produce toxins such as microcystins. These toxic compounds are not produced by Spirulina itself, but may be the result of contamination of Spirulina batches with other toxin-producing algae. For this reason, a high degree of quality control is needed to ensure that the purity of spirulina and other supplements sourced from algae is high, and are free of contamination. Also, the water supply is important when manufacturing such foodstuffs. For example, lead, mercury and arsenic contamination in the water supply has ultimately appeared in spirulina supplements manufactured in China.

Previous commercial attempts to cultivate such an algal species have attempted to replicate the same environment which results in the rapid algal “bloom” conditions. To do this, example systems comprising water tanks, or lined ponds, or various shapes of bioreactor are used, all of which require exposure to sunlight as well as access to atmospheric carbon dioxide for plant photosynthesis to occur. Such ponds and reactors may contain fresh or salt water, depending on the species of algae to be cultivated.

These previous commercial attempts to cultivate photosynthetic algae species suffer from low yields, and variations in the growth rate of the algae which can be a result of unsuitable ambient temperature, or lack of penetrating sunlight, or poor mixing of the host fluid carrying the algal spawn and the available nutrients. To make up for the low yields and poor growth rates which are often observed due to these other factors, tanks, ponds and reactors containing water and nutrients usually require a long residence time to allow the algae to be cultivated. Consequently, to produce a commercial-scale quantity of product, the commercial pond or reactor must become very large in size. This is problematic when attempting to locate a suitably large physical site location, as well as being prohibitively capital intensive.

Furthermore, once a cultivation pond or reactor becomes too large, it can then be difficult to reach some of the grown algae in the reactor for its harvesting and removal. It is undesirable to submit the algae which is grown in the reactor to vigorous pumping, as this may destroy the formed organic material, so there are some prior art examples of paddle stirrers or paddle wheels which are employed to agitate the bioreactor fluid. However, these relatively gentle means of stirring the pond or reactor are also not always sufficient to suspend the formed algal material, and to prevent it from settling in the reactor under gravity.

In certain known algal growth reactors, use has been made of a series of fluid flow channels arranged in a raceway (or maze-like, or serpentine) configuration having various algae growth regions/zones established in channels in different sections of the raceway. These growth regions/zones are achieved by the use of pumps which interconnect some of the channels to provide a recycle of at least some of the fluid flow. In practice such arrangements have many disadvantages, since algae can be destroyed by over-pumping, and the operational costs of pumping can become prohibitively high.

Algal growth reactors are also known which use a raceway configuration, but which do not continuously recirculate the flow, nor continuously harvest the biomass. In such non-recirculating systems, a relatively stable and suspended concentration of biomass all along the length of a raceway system can be achieved by moving the flow along at a minimum velocity along with dilution of the flow to allow sunlight to penetrate as well as to maintain suspension. Dilution of the biomass is achieved by making use of raceway channels sections, which are designed to become consecutively wider or deeper. Such prior art systems suffer from the disadvantages of large-scale capital equipment costs in addition to prohibitively high pump operational costs.

There remains a need for a system and method for growing biological organisms, which can be suitable for efficient organism growth at the lowest possible operation cost. In particular, for a system to be suitable for growing algae, it needs to be able to maximise carbon dioxide absorption and sunlight exposure to in turn maximise the product yield, and do so in a manner which can overcome any water contamination issues mentioned, as well as to avoid costly capital outlays and operational expenditure.

### SUMMARY OF THE DISCLOSURE

In a first aspect, embodiments are disclosed of system for cultivating one or more biological organisms, the system comprising: (a) a flow passage arranged in use for conveying a flow of a fluid containing nutrients and biological organisms suspended in the fluid; (b) a pumping device operable in use to convey the said flow through the flow passage when arranged in fluid communication therewith; and (c) a capture device operable in use to capture a portion of said flow when arranged in fluid communication therewith; wherein, during operation of the system, the configuration of the flow passage and the operation of the pumping device causes the said flow to be conveyed at a substantially uniform flowrate throughout the flow passage, and a quantity of the biological organisms which become cultivated in the said flow as a result of exposure to a source of light are captured for removal from the said flow by the capture device.

The present inventor has developed a system for cultivation of biological organisms such as algae using a unidirectional flow passage which provides exposure of the fluid to air (containing carbon dioxide) and sunlight during use. Such an elongate passage provides a sufficiently long residence time for algae to develop and to grow while the fluid is moving therealong. The present system has a pumping arrangement which moves the fluid flow through the passage so that the flowrate is substantially the same over the whole length of the passage. Thus the flow of the fluid (such as water containing dissolved and solid matter in the form of nutrients and biological organisms being suspended in the water) is substantially the same over the whole channel system. There is no requirement in the system for various regions of higher or lower speed flows, and therefore no side channels or recycle flows repeatedly exiting and then re-entering the flow passage at various points using recirculating pumping arrangements, which can lead to the destruction or disruption of the formed organic material by over-pumping it. The flowrate established in the flow passage of the present system is sufficient to keep the growing biological organisms from settling out on the base surface or floor of the flow passage.

When the term “substantially uniform flowrate” is used in this specification, this refers to a similar volumetric or mass flowrate being found at all points along the flow passage, which can encompass within its meaning some small variations in flowrate due to frictional losses, spillage and minor localised swirling or short-circuiting, for example.

In certain embodiments, the flow passage and the pumping device of the system are arranged in use to continuously recirculate the said flow therethrough. The use of a continuous recycle, closed loop system means that the biological organisms can be grown in, and harvested from, the same flow passage on a continuous basis, with the benefits of a “batch” style operation, which can minimise the risk of water contamination from unknown constituents in any newly-introduced feed flows from rivers, groundwater and the like.

In one embodiment, the flow passage comprises two flow channels, a distal end region of each flow channel arranged to be in fluid communication with a distal end region of the respective other flow channel. In one form of this, said flow channels are elongate, substantially parallel and arranged adjacent to one another, although in other forms the flow channels can be curved or of different side wall shapes to one another.

When the term “unidirectional” is used in this specification, this refers to the entire mixed flow of fluid, nutrients and biological organisms generally moving together as one, even if the actual physical directional orientation of the said flow varies, for example if it moves in one direction for a period of time, and then in another direction for a subsequent time period (such as by using the aforementioned adjacent, parallel flow channels in fluid communication with one another, in which the flow is alternately directed in one direction, and then in a 180 degrees opposite direction immediately thereafter).

In one embodiment, a plurality of said flow passages are arranged adjacent to one another, for example, in or on surrounding ground. Such a flow passage with two flow channels can be used as a standalone passage for cultivation of biological organisms such as algae, but it can also form the basic unit of a modular system which is arranged in use to have multiple such flow passages linked together, for the cultivation of biological organisms.

In one form of this, some or all of said flow passages which are arranged adjacent to one another are adapted to be placed in fluid communication with a respective other flow passage. In one particular embodiment, said adaptation is operable to cause a distal end region of a flow channel of a flow passage to be placed in fluid communication with a respective other distal end region of a flow channel of an adjacent flow passage. For example, this could be achieved in use by way of an openable channel, pipe or some other type of fluid-carrying conduit, fitted with a weir, gate valve or similar fluid control device. A pump switched on and off can also place the passages in fluid communication.

In an alternative embodiment, the flow passage comprises three or more flow channels, a distal end region of each flow channel arranged to be in fluid communication with a distal end region of a respective other flow channel. In one form of this, the flow passage comprises a series of substantially parallel, adjacent flow channels, each flow channel being in fluid communication at either end with another flow channel, resulting overall in a serpentine pattern flow passage in which the unidirectional flow is alternately directed in one direction, and then in a 180 degrees opposite direction immediately thereafter, and so on.

In one particular form of this, the flow channels are open channels which are at least partially excavated into the surrounding ground, to provide wall stability for retaining flowing water, and to provide a lower cost alternative to a flow passage made within a fully above-ground concrete structure, for example.

In certain embodiments, the source of light for establishing growth conditions is sunlight. The use of open channels allows both evaporation of water as well as the ingress of rainwater, but most importantly, uninterrupted light in the daytime for photosynthesis of the biological organisms.

In certain embodiments, the said flow throughout the flow passage is of a predetermined maximum depth and a predetermined minimum velocity.

In one form of this, the predetermined maximum depth allows the said light to penetrate substantially all the way into the said flow to optimise cultivation conditions for the biological organisms. In certain embodiments, the predetermined maximum depth is less than about 0.5 metres. In one form of this, the predetermined maximum depth is more than about 0.2 metres and less than about 0.5 metres. In one particular form of this, the predetermined maximum depth is more than about 0.2 metres and less than about 0.4 metres. One of the main problems of prior art reactors is lack of sufficient light to support photosynthesis. By using a long flow channel at such exemplary fluid depths, but with a substantially uniform flowrate, the light can penetrate the algae, and the flowrate ensures that no residual solids material remains left behind along the length of the flow passage.

In certain embodiments, the predetermined maximum depth corresponds to a quantity of solids in the form of biological organisms of less than about 4% w/v or 40 g/litre to allow said penetration of light. In one form, the predetermined maximum depth of the flow is sufficient to maintain sufficient light reaching the biological organisms which are in suspension all along the length of the flow passage.

In certain embodiments, the predetermined minimum velocity is more than about 0.3 metres/minute and less than about 1.0 metres/minute. In one form of this, the predetermined minimum velocity is more than about 0.3 metres/minute and less than about 0.5 metres/minute. In one particular form of this, the predetermined minimum velocity is more than about 0.3 metres/minute and less than about 0.4 metres/minute. In one particular form, the predetermined minimum velocity is about 0.35 metres/minute. Where the flow passage is of a fixed side-to-side width (metres), with a predetermined maximum fluid depth (metres) flowing therethrough in use, then the resulting cross-sectional area of the fluid flow (square metres), multiplied by the predetermined fluid velocity (metres/minute), calculates the substantially uniform flowrate (cubic metres/minutes) required to pass through the flow passage.

In certain embodiments, the predetermined minimum velocity corresponds to a quantity of solids in the form of biological organisms of less than about 4% w/v or 40 g/litre which can be maintained in suspension. The selection of a minimum velocity ensures that no residual solids material remains settled along the whole length of the passage.

The present disclosure therefore includes a method of arranging and sizing suitable process handling equipment to support a biological organism cultivation process. By selecting a maximum quantity of solids in the form of biological organisms which can be maintained in suspension, this determines a maximum depth of water in which the solids are suspended which will allow sufficient light to support photosynthesis at the lowest depth of the flow passage. Then the selection of a flow velocity to ensure that no residual solids material (at that solids density) can settle out under gravity in the flow passage will determine the volumetric flowrate of the water and suspended solids, assuming a fixed width of flow passage.

In certain embodiments, the capture device for removing a quantity of the cultivated biological organisms from the said flow comprises one or more of the group comprising: vacuum suction device, filtration pump, centrifuge, hydrocyclone, spiral classifier, belt filter, disc filter, filter press, and screening device. In one form, the capture device is located nearest to the end of the length of the flow passage. In the embodiment of a serpentine pattern of flow channels, the end of the flow passage occurs in the last flow channel, at which point the cultivated biological organisms, which have grown throughout their movement along the flow passage, are ready to be harvested.

During harvesting, a quantity of the cultivated biological organisms is removed by physical separation from the substantially uniform fluid flowrate by the capture device, and then in certain embodiments after such removal has occurred, a flow of residual liquid from the capture device is returned into the flow passage at a point upstream of the capture device.

In certain embodiments, the pumping device is a low solids water pump. In one form, this pump is arranged to pump the flow of residual liquid from the capture device upstream into the flow passage, and after the aforementioned removal of a quantity of the cultivated biological organisms. Because the flow of residual liquid being returned upstream contains the lowest amount of the biological organisms, it can be pumped back into the first portion of the flow passage, causing relatively little or no damage to the remaining biological organisms. In one example flow passage, this residual fluid is pumped back into a first flow channel of a series of parallel flow channels of a serpentine flow passage; or, in another example flow passage having dual, elongate, substantially parallel and adjacent channels, this residual fluid is pumped back from an end region of the second flow channel into the start region of the first flow channel. In the present exemplary system, it is this same pumping device which serves to move the fluid flow through the channels at a flowrate which is substantially the same over the whole length of the flow passage. No other pumping arrangement is needed in the remainder of the flow passage where the biological organisms are being cultivated, thus minimising disruption from pumping or other agitation.

In certain embodiments, a flocculant is added to the flow at a location upstream of the capture device, to assist in aggregation and removal of the cultivated biological organisms. In certain embodiments, the biological organisms comprise algae.

In a second aspect, embodiments are disclosed of a method for cultivating one or more biological organisms, the method comprising the steps of: (a) arranging a pumping device to be in fluid communication with a fluid located in a fluid passage, the fluid containing an amount of nutrients and biological organisms suspended therein; (b) conveying a flow of the said fluid through the flow passage by operation of the pumping device; and (c) capturing a portion of the said flow by operation of a capture device when it is arranged in fluid communication with the said flow; wherein, the step of operating the pumping device causes the said flow to be conveyed at a substantially uniform flowrate throughout the flow passage, and a quantity of the biological organisms which become cultivated in the said flow as a result of exposure to a source of light are captured for removal from the said flow by the step of operating the capture device.

This method of cultivation of biological organisms such as algae using a flow passage which exposes of the fluid to air and sunlight provides a sufficiently long residence time for algae to develop and to grow while the fluid is moving therealong. In the present method, the fluid flow is moved through the passage so that the flowrate is substantially the same over the whole length of the passage, and this pumping arrangement is sufficient to keep the growing biological organisms from settling out on the base surface or floor of the flow passage, and to mimimise the destruction or disruption of the formed organic material.

In certain embodiments of the method, the pumping device is operated continuously, thereby creating a continuous recirculation of said flow throughout the flow passage and the pumping device. The use of a continuous recycle, closed loop system means that the biological organisms can be grown in, and harvested from, the same flow passage on a continuous basis, with the benefits of a “batch” style operation, which can minimise the risk of water contamination from unknown constituents in any newly-introduced feed flows from rivers, groundwater and the like.

In one form of the method, the operation of the pumping device is arranged to convey said flow throughout the flow passage at a predetermined maximum depth and at a predetermined minimum velocity.

In certain embodiments of the method, the predetermined maximum depth is as defined in the system of the first aspect. By using a long flow channel at such exemplary fluid depths, but with a substantially uniform flowrate, the light can penetrate the algae, and the flowrate ensures that no residual solids material remains left behind along the length of the flow passage.

In certain embodiments of the method, the predetermined minimum velocity is as defined in the system of the first aspect. The selection of a minimum velocity by the operator ensures that no residual solids material remains settled along the whole length of the passage.

In certain embodiments of the method, the step of capturing and removing a quantity of the cultivated biological organisms from the said flow comprises operating one or more of the group comprising: vacuum suction device, filtration pump, centrifuge, hydrocyclone, spiral classifier, belt filter, disc filter, filter press, and screening device. In one form, the operator locates the capture device nearest to the end of the length of the flow passage. Whether the flow passage is made up of a series of parallel flow channels of a serpentine flow passage, or, in another simpler example, the flow passage has just two, elongate, substantially parallel and adjacent channels, the end of the flow passage occurs in the distal end region of the last connected flow channel, at which point the cultivated biological organisms, which have grown throughout their movement along the flow passage, are ready to be harvested. In one example of the flow passage having dual elongate, adjacent channels, this residual fluid moves from an end region of the second flow channel, via a conduit across the channel wall, and into the body of fluid located in the start region of the first flow channel.

During harvesting, the operator uses the capture device to separate a quantity of the cultivated biological organisms from the substantially uniform fluid flowrate, and then in after such removal has occurred, the operator directs a flow of residual liquid from the capture device to be returned into the flow passage at a point upstream of the capture device. Because the flow of residual liquid being returned upstream contains the lowest amount of the biological organisms, it can be returned into the flow passage by pumping, causing relatively little or no damage to the remaining biological organisms (in one example, into a first flow channel in a flow passage formed by a pair of elongate, substantially parallel and adjacent channels; in another example, into the first of a series of parallel flow channels of the serpentine flow passage).

In certain embodiments, the method further comprising the step of an operator controlling the addition of a flocculant to the flow at a location upstream of the capture device, to assist in aggregation and removal of the cultivated biological organisms by the capture device.

In a third aspect, embodiments are disclosed of an apparatus for cultivating one or more biological organisms, the apparatus comprising: (a) a flow passage comprising two or more flow channels arranged in use for conveying a flow of a fluid containing nutrients and biological organisms suspended in the fluid; and (b) a pumping device associated with the flow passage, said pumping device being operable to convey said flow of fluid through the flow passage when arranged in fluid communication therewith in a first operating configuration, wherein, in a second operating configuration, said flow passage is adapted to be placed in fluid communication with at least one further flow passage, and said pumping device is operable to convey said flow of fluid through the flow passage and the further flow passage(s).

The present inventor has developed a modular system for cultivation of biological organisms such as algae using a unidirectional flow passage which provides exposure of the fluid to air (containing carbon dioxide) and sunlight during use. Such a modular apparatus provides a simple way to adjust the residence time available for algae to develop and to grow, while the fluid is moving therethrough. As has been explained earlier, for simplicity of operation, as well as to prevent over-pumping and destruction of the product, or settling out of the algae, the fluid flow can be moved through the passage at a flowrate which is substantially the same over the whole length of the passage. To give an added degree of flexibility to the algae growth process, the flow passage of the present aspect can also be configured to comprise any number of units of two-channel flow passages arranged in series.

In certain embodiments, the pumping device is operable to continuously recirculate the said flow through each of the flow passages, if the apparatus is in the second operating configuration. The use of a continuous recycle, closed loop system means that the biological organisms can be grown in, and harvested from, any one of the flow passages on a continuous basis, with the benefits of a “batch” style operation.

In certain embodiments, a distal end region of each flow channel is adapted to be in fluid communication with a distal end region of another flow channel. In one embodiment, the flow passage comprises two flow channels which are elongate, substantially parallel and arranged adjacent to one another. Such a flow passage with two flow channels can be used as a standalone passage for cultivation of biological organisms or it can also form the basic unit of a modular system which is arranged in use to have multiple such flow passages linked together in use.

In other forms the flow channels can be curved or of different side wall shapes to one another. In yet other embodiments, each flow passage can comprise more than two flow channels. For example, the flow passage may comprise three or four substantially parallel, adjacent flow channels, each flow channel being in fluid communication at either end with another flow channel.

In certain embodiments, the said flow passage adaptation is operable to cause a distal end region of a flow channel of a flow passage to be placed in fluid communication with a respective other distal end region of a flow channel of the further flow passage. This allows some or all of said flow passages which are arranged adjacent to one another, to effectively form a continuous system for the cultivation of biological organisms such as algae in a unidirectional flow passage which provides exposure of the fluid to air (containing carbon dioxide) and sunlight during use.

The ability to link (and indeed to isolate) separate growth passages also gives an operator great flexibility to choose a sufficiently long residence time for a particular type algae or other biomass product to develop and to grow while the fluid is moving therethrough. For example, a fast-growing type of algae may only need to flow through two flow passages (each flow passage comprising two flow channels) before harvesting can occur. In other cases, a slower growing product may need to be developed over a larger number of connected flow passages. The flow passages are typically linked in use by way of an openable channel, pipe or some other type of fluid-carrying conduit, fitted with a weir, gate valve or similar fluid control device such as an actuatable pump.

In certain embodiments, a capture device is operable in use to capture a portion of said flow when arranged in fluid communication therewith and removing a quantity of the cultivated biological organisms from the said flow. Such a device can be one of the group comprising: vacuum suction device, filtration pump, centrifuge, hydrocyclone, spiral classifier, belt filter, disc filter, filter press, and screening device. This device can be positioned at any of the flow chambers.

In certain embodiments, the apparatus of the third aspect is otherwise as defined by the features of the system of the first aspect.

In certain embodiments, the apparatus of the third aspect is otherwise operable using the steps of the method of the second aspect.

Other aspects, features, and advantages will become apparent from the following detailed description when taken in conjunction with the accompanying drawings, which are a part of this disclosure and which illustrate, by way of example, principles of the inventions disclosed.

### DESCRIPTION OF THE FIGURES

The accompanying drawings facilitate an understanding of the various embodiments which will be described:

FIG. 1 is a perspective side view of a portion of a flow passage in the form of open channels, used for a system of cultivating biological organisms, in accordance with a first embodiment of the present disclosure;

FIG. 2 is a perspective, top view of a portion of the flow passage in the form of open channels, according to FIG. 1;

FIG. 3 is a further perspective, top view of the flow passage in the form of open channels, according to FIG. 1;

FIG. 4 is a further perspective, top view of a portion of the flow passage in the form of open channels, according to FIG. 1, along with an adjacent work area for location of a capture device used for biological organism harvesting from the flow passage;

FIG. 5 is a view of a quantity of some biological organisms in the form of algae, after physical removal from the flow passage according to FIG. 1 by use of the capture device;

FIG. 6 is a perspective, top view of a portion of the flow passage in the form of an open channel according to FIG. 1, in which a fluid flow containing nutrients and some biological organisms and top-up water is added into the first channel of the flow passage, along with a flow of fluid which is recirculated into the first channel of the flow passage following the step of harvesting biological organisms from the fluid flow using the capture device;

FIG. 7 is a perspective, top view of an algae culturing facility used to create an algae or biomass culture prior to introduction of such material into the flow passage according to FIG. 1;

FIG. 8 is a schematic plan view of the flow passage, according to FIG. 1;

FIG. 8B is a schematic, sectional end elevation view of the flow passage according to FIG. 8 when viewed along the sectional line B-B;

FIG. 8C is a schematic end elevation view of the flow passage according to FIG. 8 when viewed in the direction of arrow C;

FIG. 9 is a perspective side view of a portion of a flow passage in the form of two open channels, used in a system of cultivating biological organisms, in accordance with a second embodiment of the present disclosure;

FIG. 10 is a perspective, top view of a portion of the flow passage in the form of two open channels, according to FIG. 9;

FIG. 11 is a schematic plan view of the flow passage, according to FIGS. 9 and 10;

FIG. 11B is a schematic, sectional end elevation view of the flow passage according to FIG. 11 when viewed along the sectional line BB-BB;

FIG. 11C is a schematic end elevation view of the flow passage according to FIG. 11 when viewed in the direction of arrow CC;

FIG. 11D is a schematic side elevation view of the flow passage according to FIG. 11 when viewed in the direction of arrow DD; and

FIG. 12 is a schematic plan view of the flow passage according to FIG. 11, shown when connected to a series of adjacent, like flow passages also according to FIG. 11.

FIG. 13 shows measurements of the growth rate of algae (g/L/hr) as a function of the velocity (m/s) of a fluid as it is passed through a system for cultivating biological organisms, in accordance with the embodiment of the present disclosure shown in FIGS. 9, 10 and 11; the Figure also shows pump power consumption (Watts) as a function of the velocity (m/s) of the same fluid;

FIG. 14 shows measurements of the growth rate of algae (g/L/hr) as a function of the flowrate (L/hr) of a fluid as it is passed through a system for cultivating biological organisms, in accordance with the embodiment of the present disclosure shown in FIGS. 9, 10 and 11; the Figure also shows pump power consumption (Watts) as a function of the velocity (m/s) of the same fluid; and

FIG. 15 shows measurements of the growth rate of algae (g/L/hr) as a function of the depth (m) of a fluid as it is passed through a system for cultivating biological organisms, in accordance with the embodiment of the present disclosure shown in FIGS. 9, 10 and 11.

### DETAILED DESCRIPTION

This disclosure relates to the features of a system and method for cultivating a biomass such as algae, in a flow passage in which a fluid is pumped therearound, and which is exposed to sunlight to facilitate growth of the algae.

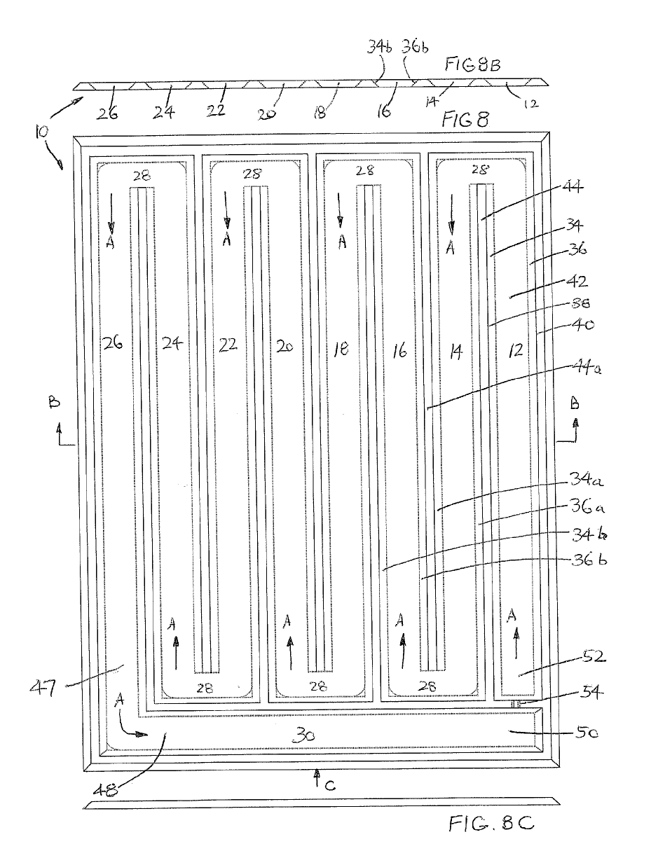

Referring to the drawings, and particularly to FIG. 8, a flow passage 10 is shown in the form of eight successively linked, substantially parallel, adjacent, open top channels arranged in a serpentine configuration (shown with part numbers 12, 14, 16, 18, 20, 22, 24 and 26). With the exception of the first channel 12, where only one distal end has the same arrangement, for each of the other seven channels 14, 16, 18, 20, 22, 24 and 26, each distal end of the said channels is in direct flowing fluid communication with another, immediately adjacent channel, via a respective, short, laterally oriented flow channel portion (each one numbered 28 in the drawing).

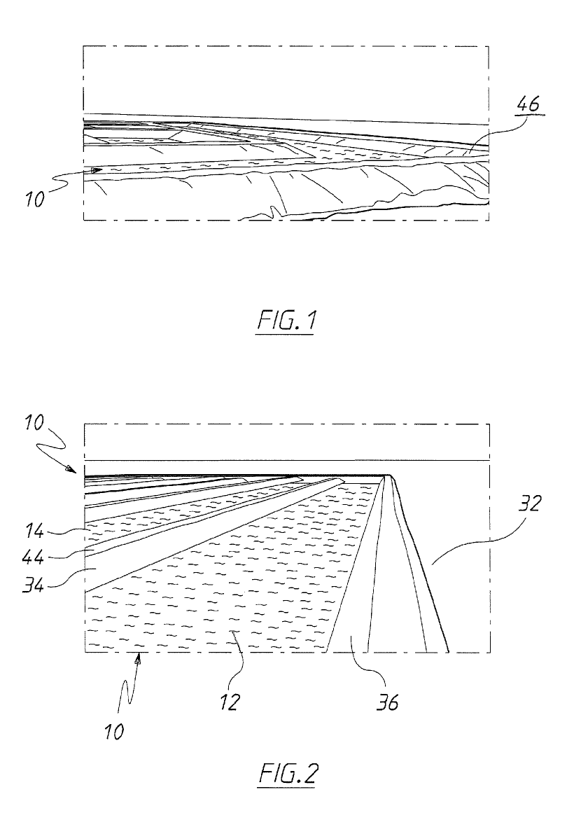

The serpentine configuration flow passage 10 is comprised of open top flow channels 12, 14, 16, 18, 20, 22, 24, 26 which are at least partially excavated into the surrounding ground 32, a feature which is more clearly shown in FIGS. 1, 2, 3 and 4. The opposing elongate inner side walls 34, 36 of each of the channels 12, 14, 16, 18, 20, 22, 24, 26 are inclined inwardly when moving down from the uppermost surface edge 38, 40 of the channel side walls 34, 36, to taper in a downward direction to meet the flat floor base 42 of the channels 12, 14, 16, 18, 20, 22, 24, 26. Apart from the outermost channels 12 and 26, the opposing elongate side walls of each channel of the flow so passage (for example, walls 34a, 36a of channel 14) are arranged back-to-back with another respective elongate side wall of an adjacent channel (for example walls 34b, 36b of channel 16) to form a stabilised barrier wall (for example barrier wall 44a having tapered sides 34a, 36b) for retaining flowing water within each of the open top channels 12, 14, 16, 18, 20, 22, 24, 26.

Arranging the channels 12, 14, 16, 18, 20, 22, 24, 26 partially within the surrounding ground 32 provides a lower cost alternative to a flow passage which is fully constructed above-ground, such as a concrete walled structure, for example. Each of the eight elongate channels 12, 14, 16, 18, 20, 22, 24, 26, as well as the short, laterally oriented flow channel portions 28, can be formed by excavation of the surrounding ground 32 made in rows, followed by the piling up of the excavated earth in the space between those rows, and then sealing and stabilising those earth piles with a roadwork polymer, for example, to form inclined inner side walls 34, 36 of the barrier wall 44. Finally, the inclined inner side walls 34, 36 and the flat floor base 42 of each channel 12, 14, 16, 18, 20, 22, 24, 26, are covered with a UV-resistant, continuous, water impermeable barrier material, such as thick PVC sheeting 46 (shown having a white colour in the drawings, which is advantageous because of the sunlight reflective properties of this colour). This barrier sheeting 46 ensures that the water flowing within the serpentine flow passage 10 does not escape from the channels 12, 14, 16, 18, 20, 22, 24, 26, for example by leakage into surrounding ground 32, but is recirculated continuously, as will be described.

In other embodiments, the inner side walls of the channels can be sloped at other angles compared to that shown in the drawings, and in yet further embodiments, the inner side walls can also be vertically oriented, and formed from concrete or other materials. The inner surfaces of any of these embodiments of channel can be coated with an adhering sealant compound to form a water-impermeable barrier, or fitted with the PVC sheeting as outlined above.

With reference to FIG. 8, the serpentine flow passage 10 also includes a further open top channel 30 which is arranged in a lateral orientation to each of the other open top channels (12, 14, 16, 18, 20, 22, 24 and 26), but this lateral open top channel 30 is only in direct, flowing fluid communication at its distal end 48 with the adjacent distal end 47 of channel 26. In all other respects, the lateral open top channel 30 is similar in elongate inner side wall configuration to the other open top channels (12, 14, 16, 18, 20, 22, 24 and 26), and its method of construction (by excavation, sealing etc) is carried out in a similar manner as has been described for the other open top channels (12, 14, 16, 18, 20, 22, 24 and 26).

In use, the lateral open top channel 30 forms the last stage of the serpentine flow passage 10. Its other distal end 50 is in proximity to (but not in direct flowing fluid contact via a channel with) the distal end 52 of the first channel 12. The distal end 50 of the lateral open top channel 30 is the general location where the physical harvesting of cultivated biological organisms occurs in the flow passage 10, as will be described.

In one non-limiting, exemplary embodiment, the lateral width of each of the eight parallel open top channels 12, 14, 16, 18, 20, 22, 24, 26, and of the laterally oriented open top channel 30, as measured from the uppermost surface edge 38, 40 of each of the opposing channel side walls 34, 36, is 5 metres. The lateral width between the opposed vertical side walls of the short, laterally oriented flow channel portions 28 is also 5 metres. The end-to-end length of each of the parallel open top channels 12, 14, 16, 18, 20, 22, 24, 26 is about 50 metres, and the end-to-end length of the laterally oriented open top channel 30 is about 40 metres. An exemplary average residence time in the whole flow passage 10 for fluid which contains suspended biological organisms and algae, is somewhere of the order of 24 hours to 72 hours.

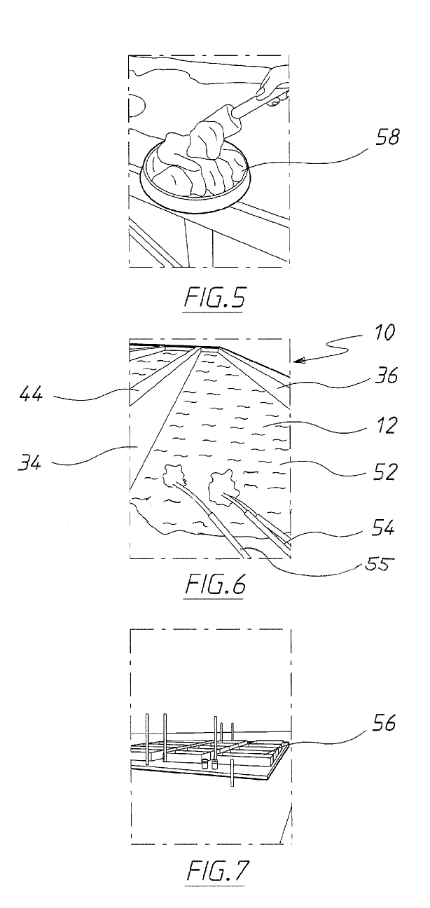

In use, to begin the process of cultivating biological organisms, the serpentine flow passage 10 is partially filled with water, which in use will be recirculated in a forward flow direction through each of the channels of the flow passage 10, in sequence through the parallel open top channels 12, 14, 16, 18, 20, 22, 24 and 26, and then into the laterally oriented open top channel 30, following the arrows labelled ‘A’, shown in FIG. 8. The start point of the cultivation process is shown in FIG. 6, at the distal end 52 of channel 12, at which point which nutrients (such as liquid fertiliser) and cultured biological organisms are added into the flow via hose 55 and suspended therein. The water at this point does not contain a large quantity of any cultivated organisms, such as algae. The pure cellular cultures are obtained from an approved source and are then inoculated in separate culture tanks 56 (as shown in FIG. 7) for a few weeks to become established, prior to use in the flow passage 10.

A low solids water pump (not shown) which is normally seated outside of the flow passage 10 on the surrounding ground 32, in use carries a flow of water out of the distal end 50 of the laterally oriented open top channel 30, and then passes that flow into the adjacent distal end 52 of the channel 12 via a hose 54 (as shown in FIGS. 6 and 8). Any type of conduit or even a fixed pipe connection system can also be used instead of the hose 54. The forward flow of the water passes in sequence through each of the respective open top channels 12, 14, 16, 18, 20, 22, 24 and 26 of the serpentine flow passage 10, in the direction of arrow ‘A’ (FIG. 8), and back to the laterally oriented open top channel 30, from which is then recirculated back into to the channel 12, and so on. As the water in the system recirculates, the combination of added nutrients, atmospheric carbon dioxide, sunlight and agitation of the fluid flow encourages the growth cultivation of the biological organisms which are continually suspended in the flowing water. The unidirectional flow of the water along the entire flow passage 10 is driven by a low solids water pump (not shown), which conveys the said flow of water and suspended solids at a substantially uniform flowrate throughout the flow passage 10. The flow of water and suspended solids can continue to be recirculated continuously in this closed loop system, which means that the biological organisms are given sufficient residence time to grow, and then be harvested from, the same flow passage 10 on a continuous basis, but using what is essentially a “batch” style cultivation operation which has the benefits of product purity and water quality control.

As the flow of water reaches the distal end 52 of the laterally oriented open top channel 30, and because the cultivation process has taken hold in the liquid flow by this point, a quantity of solids in the form of biological organisms of up to but less than about 4% w/v or 40 g/litre is being maintained in suspension, and is ready for removal from the liquid. To avoid blockages, or other operational problems, it is undesirable for residual biological solids material to settle somewhere along the length of the flow passage 10, for example on the floor base 42 of the channels 12, 14, 16, 18, 20, 22, 24 and 26. The effect of the unidirectional flow at a substantially uniform flowrate acts to prevent such settling from occurring, as well as to avoid ‘overpumping’ of the formed biological organisms, which can lead to disruption or even destruction of the formed organisms.

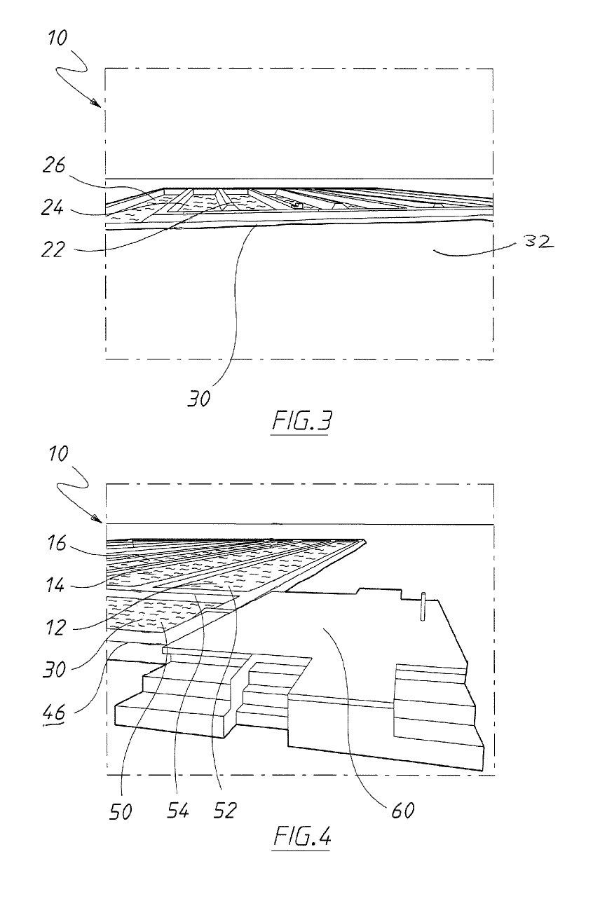

At the distal end 52 of the open top channel 30, a capture device in the form of a suction extraction and filtration system can be positioned in contact with the flow in the lateral open top channel 30 and then operated by a user to remove some of the liquid, while also extracting (or harvesting) the solid biological organisms which are present in suspension. The filtration system can also involve the use of the low solids water pump, in combination with some sort of screen, filter or other separation membrane, arranged on the work platform 60 shown in FIG. 4, to capture the biological solids material, after which the filtration system releases the extracted water back upstream into the distal end 52 of the flow channel 12 via the low solids water pump, as mentioned previously, and as shown in FIGS. 4 and 6.

In alternative embodiments, the capture and separation step can be achieved by gravity separation or even by centrifugal or cyclonic apparatus, for example a small scale Alfa Laval-brand centrifuge, to dewater the algae product. An operator can also add specific reagents at this point which may assist in the separation of solids from liquids, for example using coagulants or flocculants. The separated filter cake of moist solids is then removed from the separation device for further drying, and a typical example of moist solids sludge 58 is shown in FIG. 5. The filtered and dried solids material will later be subjected to other processing steps such as purification, classification and packaging and so on, prior to commercial use.

The operator of the system is required to maintain a fluid and suspended solids flow throughout the flow passage 10 of a predetermined maximum depth and a predetermined minimum velocity. The predetermined maximum depth is sufficient to allow light to penetrate substantially all the way into the flow in the flow passage 10, to optimise photosynthetic cultivation conditions for the biological organisms. A usual fluid depth for this to occur is less than about 0.5 metres and more than about 0.2 metres, for the type of biological solids loading of algae (less than about 4% w/v or 40 g/litre) which is suspended in the exemplary flow. Equipment for automated monitoring of flow depth and turbidity is available, as are responsive control systems for adjusting these flow parameters by adding or removing water as required.

The predetermined minimum velocity needs to be sufficient to maintain the solid biological organisms present in the fluid in suspension without settling, and a usual range for this to occur is a velocity of more than about 0.3 metres/minute and less than about 1.0 metres/minute for the expected biological solids loading of algae (that is, less than about 4% w/v or 40 g/litre) which is suspended in the exemplary flow. The parameters of flow depth, velocity and channel width will determine the substantially uniform flowrate (cubic metres/minutes) which is required to pass through the flow passage 10, and this value will, in turn, determine the volumetric capacity of the low solids water pump(s) used in the step of capturing and separating the biological solids material from the water flow in the lateral open top channel 30, after which the water is returned upstream into the flow channel 12. Equipment for monitoring of volumetric flowrate is available, as are responsive control systems for adjusting pump speed, as required.

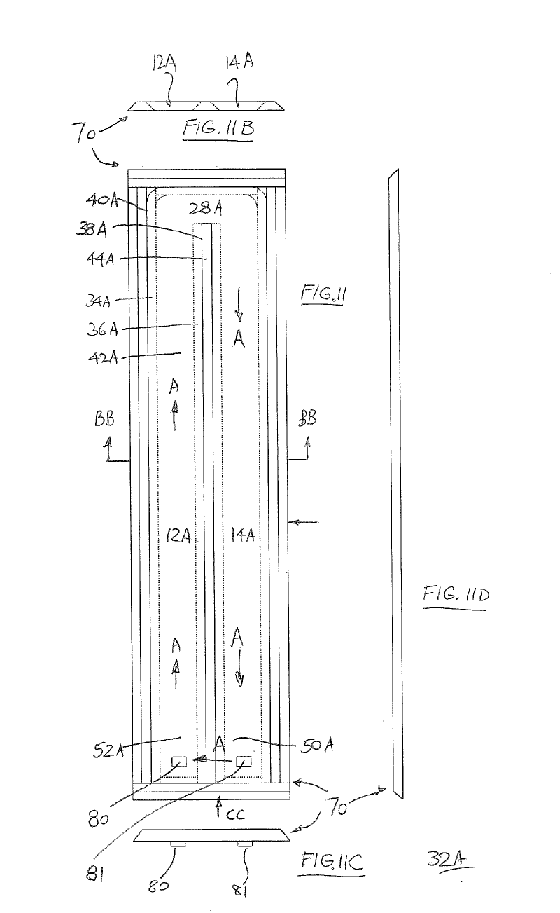

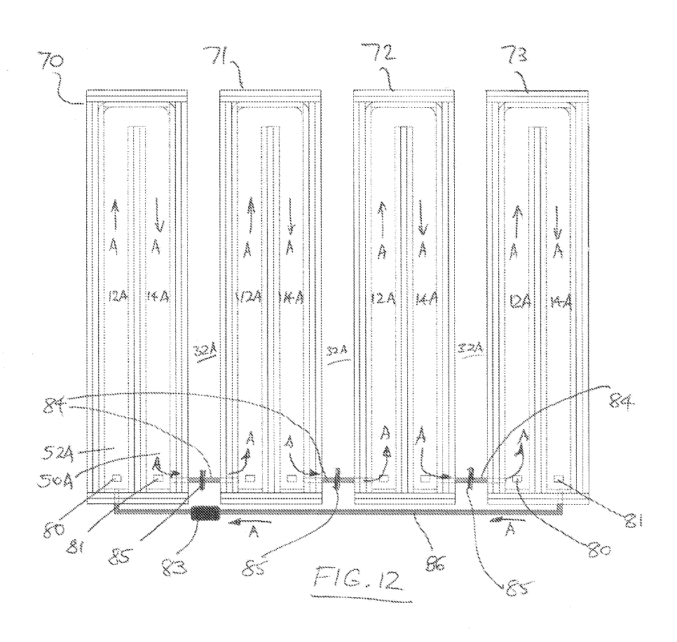

Referring to the drawings, and particularly to FIG. 11, a flow passage 70 is shown in the faun of two successively linked, substantially parallel, adjacent, open top channels (shown with part numbers 12A and 14A). In all respects these channels are similar to the channels 12, 14, 16, 18, 20, 22, 24 and 26, which have been previously described in relation to the embodiment of the algae cultivation system shown earlier in FIG. 8, for example.

To avoid unnecessary repetition, in this specification like parts having like functionality to earlier examples or embodiments which have already been described shall be given the same part number, but with the addition of the letter “A” thereafter.

In use, the open top channel 14A forms the last stage of the flow passage 70. Its distal end 50A is in proximity to (but not in direct flowing fluid contact via a channel with) the distal end 52 of the first channel 12A. The distal end 50A of the open top channel 12A is the general location where the physical harvesting of cultivated biological organisms occurs in the flow passage 70.

In one non-limiting, exemplary embodiment, the lateral width of each of the open top channels 12A, 14A, as measured from the uppermost surface edge 38A, 40A of each of the opposing channel side walls 34A, 36A, is 5 metres. The end-to-end length of each of the parallel open top channels 12A, 14A is about 50 metres. The average residence time in the whole flow passage 70 for fluid which contains suspended biological organisms and algae, is much shorter than for the previous serpentine embodiment described.

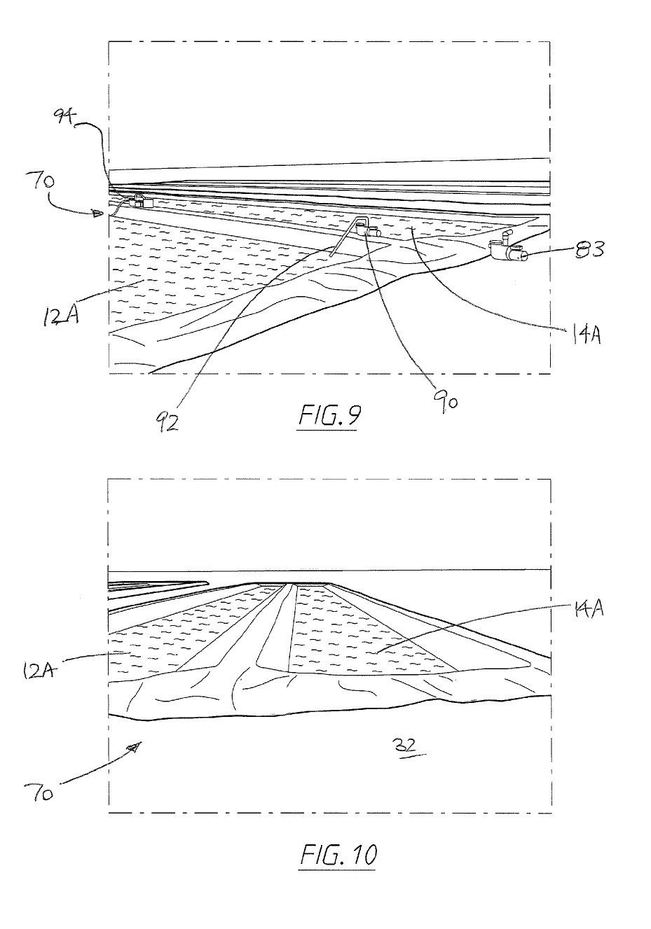

The process of cultivating biological organisms, is similar to the previous description given in relation to flow passage 10. To maintain recirculation, a low solids water pump 90 (FIG. 9) is seated on a wall of the flow passage 70 (or can be on the surrounding ground 32A), and is arranged in use to extract a flow of water out of the distal end 50A of the open top channel 14A, and then to discharge that flow into the adjacent distal end 52A of the channel 12A via hoses 92. It may even be possible in other installations to interconnect the two channels 12A, 14A by way of an underground pipe which is connected to the floor sumps 80, 81 located at the respective distal ends 52A, 50A of the channels 12A, 14A.

The forward flow of the water passes in sequence through the respective open top channels 12A and 14A of flow passage 70, in the direction of arrow ‘A’ and then is then recirculated back into to the channel 12A, and so on. As the water in the system recirculates, the combination of added nutrients, atmospheric carbon dioxide, sunlight and agitation of the fluid flow encourages the growth cultivation of the biological organisms which are continually suspended in the flowing water. The unidirectional flow of the water along the flow passage 70 driven by the low solids water pump 90, conveys the flow of water and suspended solids at a substantially uniform flowrate throughout the flow passage 70. The flow of water and suspended solids can continue to be recirculated continuously in this closed loop system, so that the biological organisms are given sufficient residence time to grow, and then be harvested from, the same flow passage 70 on a continuous basis, using what is essentially a “batch” style cultivation operation which has the benefits of product purity and water quality control.

At the distal end 50A of the open top channel 14A, a capture device in the form of a suction extraction and filtration system 94 can be positioned in contact with the flow and then operated by a user to remove some of the liquid, while also extracting (or harvesting) the solid biological organisms which are present in suspension, after which the filtration system releases the extracted water back upstream into the distal end 52A of the flow channel 12A, as mentioned previously.

Such a flow passage 70 with two flow channels 12A, 14A can be used as a standalone passage used as part of a system for cultivation of biological organisms, or it can also form one basic unit of a modular system which is arranged in use to have several such flow passages linked together in use, as will now be described.

Referring now to FIG. 12, four identical flow passages 70, 71, 72, 73 are shown arranged in series for conveying a flow of a fluid containing nutrients and biological organisms suspended in that fluid, from one to the next flow passage 70, 71, 72, 73, in a flow direction which is also indicated by arrow “A”. The fluid in this system is conveyed by means of a pump 83, which is arranged to be in fluid communication with all four flow passages 70, 71, 72, 73, and which is located on a fluid return line 86 which extends via hoses from the sump 81 outlet of the channel 14A of flow passage 73, back to the sump 80 inlet of the channel 12A of flow passage 70. Each of the four flow passages 70, 71, 72 and 73 are also linked to an immediately adjacent flow passage 70, 71, 72 and 73 by means of a length of a pipe or hose 84, which extends therebetween via pipe connections made to low solids water pumps 85 which are seated on the ground 32A between respective flow passages 70, 71, 72, 73, and which are arranged in use to extract a flow of water out of the distal end 50A of the open top channel 14A, and then to discharge that flow into the adjacent distal end 52A of the channel 12A of the next adjacent flow passage, via hoses 92. It may even be possible in other installations to interconnect the channels by way of an underground pipe which is connected to the floor sumps 80, 81 located at the respective distal ends 52A, 50A of the channels 12A, 14A.

In further embodiments, any number of these two-channel flow passages 70, 71, 72, 73 can be placed in fluid communication by actuating the pumps 85 and the pump 83 on the return line 86. These pumps can be continuously operated to recirculate the flow of liquid and nutrients through each of the flow passages 70, 71, 72, 73, from one to the next, so that the flow passages 70, 71, 72, 73 can operate in series, and effectively become stages of a single algae growth reactor. If a continuous recycle, closed loop system is configured by a user, then biological organisms can be grown in, and harvested from, any of the flow passages 70, 71, 72, 73 on a continuous basis.

In an algae farming operation, the ability to grow a wide range of different strains of algae at the same time is useful in order to meet customer demand. An animal stock feed may comprise a blend of various different algae products, for example those which contains either high oils, high proteins or high carbohydrates. Certain of these are fast-growing, and others may take longer to develop.

The use of a modular system for cultivation of biological organisms using a unidirectional flow passage provides a simple way for a user to adjust the residence time available for a particular algae type to grow, while the fluid is moving therethrough. Certain algae products may only need to grow by recirculation of the liquid, spawn and nutrients using two interconnected flow passages (each flow passage comprising two flow channels) for a short period of time before harvesting can occur. In other cases, a slower growing product may need to be developed over a larger number of connected flow passages. The present reconfigurable apparatus gives the user the ability to cultivate different products separately, and simultaneously. It also allows the user to ramp up production of certain algaes quite quickly, compared with prior art operations in which the whole facility would need to be stopped in order to switch over to growing a new product. None of the prior art systems have such flexibility to operate with a wide variety of algae species.

### EXPERIMENTAL RESULTS

Experimental results for the cultivating biological organisms have been produced by the inventor using the embodiment of a flow passage in accordance with FIGS. 9 to 11 of the present disclosure. In this flow passage, the two successively linked, substantially parallel, adjacent, open top channels had a lateral width of 5 metres, and an end-to-end length of each of the parallel open top channels of about 50 metres.

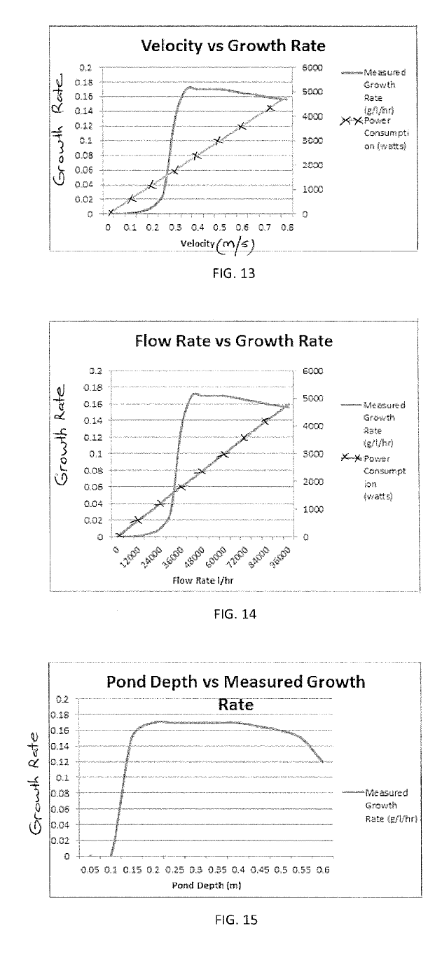

FIG. 13 shows measurements of the growth rate of algae (g/L/hr) as a function of the velocity (m/s) of a fluid as it is passed through this system. FIG. 13 also shows pump power consumption (Watts) as a function of the velocity (m/s) of the same fluid.

FIG. 14 shows measurements of the growth rate of algae (g/L/hr) as a function of the flowrate (L/hr) of a fluid as it is passed through this system. FIG. 14 also shows pump power consumption (Watts) as a function of the flowrate (L/hr) of the same fluid.

The results indicate that there is a velocity (of liquid and suspended solids) in the flow passage at which growth rate of algae is optimised and then experiences a plateau, so there is no benefit in increasing fluid velocity (and resultant power consumption) beyond that point. This minimum velocity (of around 0.4 m/s) appears sufficient to maintain the solid biological organisms present in the fluid in suspension without settling, for the expected biological solids loading of algae (that is, less than about 4% w/v or 40 g/litre). Given a fairly constant fluid cross-section and depth within the channels of the flow passage, it is also therefore observed that the optimum growth rate of algae corresponds to a liquid flowrate of around 40,000 L/hr.

The results also indicate that as the velocity and flowrate used in the flow passage was further increased, the growth rate plateau began to drop, which may indicate that the algae was ultimately being destroyed by over-pumping, as well as the operational costs of pumping becoming prohibitively high.

FIG. 15 shows measurements of the growth rate of algae (g/L/hr) as a function of the depth (m) of a fluid as it is passed through this system for cultivating biological organisms.

The results indicate that a fluid depth which is either too shallow or too deep is sub-optimal for the growth rate of algae. When the pond depth is very shallow, the flow of fluid is most likely too turbulent due to surface forces along the floor of flow passage, and algae growth is disturbed. When the pond depth is too deep, it is likely that this does not allow light to penetrate substantially all the way into the flow into the flow passage to optimise photosynthetic cultivation conditions for the biological organisms. A fluid depth of somewhere between 0.2 and 0.5 metres is ideal for the expected biological solids loading of algae in the present example (that is, less than about 4% w/v or 40 g/litre).

The inventor has developed a system and method for cultivating algae, which uses a raceway flow passage, comprising one, two or multiple interconnected elongate channel modular system, in which a fluid is pumped therethrough, which has resulted in many operational advantages over the known prior art.

In one prior art example depicted in WO2014/197919, over-pumping and destruction of the algae was the practical result of the use of the claimed algae growth reactor configuration, a prior art system which the present applicant is aware has failed technically and commercially in use. Another factor in that commercial failure was the operational costs of pumping proved to be prohibitively high. The aim of the prior art algal growth reactor was to provide inputs of biological materials (such as spawn) and water flow at the start of the flow stream, and to remove algae from the end region (or harvest zone) of the same water flow stream—in other words, for optimal operation of such an “end to end” system, the algae needs to be formed completely, and be ready to harvested by the time it passes through the entire system and reaches the harvest zone for physical removal. At that point, the water remaining in the flow stream is then pumped through a long, thin conduit, right back to the start of the flow stream on the far side of the maze reactor.

The patent applicant in WO2014/197919 attempted to reduce algae formation reactor size by utilising a fluid flow passage having a raceway (or maze-like, or serpentine) configuration which had various algae growth regions/zones established over various channels of the raceway. An arrangement of interconnecting pumps was used to provide certain parts of the raceway with a predetermined level of recycle of the fluid flow, so as to create reactor operating zones (being one or two growth zones, and a harvesting zone). The recycling of the fluid using pumps aimed to achieve specific residence times of fluid and algae in the various reactor growth zones, depending on the requirements. For example, a high recycle ratio in the first stage of the raceway gave a sufficient residence time for rapid, early stage growth of the algae. However, the over-pumping of the water-algae flow in that same zone also had the effect of physically destroying the grown algae, in addition to prohibitively high pump running costs.

This same prior art system also suffered from the issue that if the algae was not formed completely and ready to harvest by the time it reached the harvest zone, there was no opportunity to allow further growth to occur, for example by returning the semi-formed plant material and liquid back into the growth zone(s) of the raceway without being pumped via the long, thin conduit back around the perimeter of the raceway to the start of the flow stream. For example, over certain days when the sunlight becomes limited (such as overcast or stormy weather), it is then necessary for the flowrate of algae and liquid in the prior art system to be reduced, to compensate for expected the slower algae growth rate due to low light. Of course, a lower flow may reach the point where the velocity of the liquid becomes insufficient to suspend the partially formed algae, and such solids become settled out in the channels, thus blocking the raceway. The other possible way to maintain the algae in suspension in the raceway would be to increase the rate of flow of the recycle pumps, but this also has negative operational consequences, which have already been described. Controlling the operation of this prior art raceway system in response to the weather is fraught with difficulties.

Algal growth reactors are also known which use a raceway configuration, but which do not continuously recirculate the flow, nor continuously harvest the biomass. In such non-recirculating systems, a relatively stable and suspended concentration of biomass all along the length of a raceway system can be achieved by moving the flow along at a minimum velocity. However, the algae biomass can grow quickly as it moves along the path of the raceway.

In a non-recirculating raceway which has a fixed channel size (width and depth), to keep the photosynthetic algae growing at an optimum rate, dilution of the flow can help sunlight to penetrate the flow, as well as to ensure that sufficient water is present so that the ever-increasing mass of algae can remain in suspension. When dilution of the flow is necessary, rather than increasing the flow velocity (which can increase turbulence, which is unsuitable for algal growth), in this type of raceway system, the dilution of the biomass can also be achieved without changing flow velocity by instead making use of raceway channels sections which become consecutively wider or deeper.

Such prior art systems suffer from the disadvantages of large-scale capital equipment costs in addition to prohibitively high pump operational costs.

The present inventor has now developed a system and method for cultivating algae, in a flow passage in which a fluid is pumped therearound, having at least some of following beneficial features and outcomes:
The use of a unidirectional, open channel flow passage provides exposure of the moving fluid to air and sunlight during use, and gives a sufficiently long residence time for algae or other biological organisms to develop and to grow;
The use of an open channel flow passage allows an operator to set the fluid depth so that light can penetrate the flow to optimise cultivation photosynthetic conditions;
By pumping the fluid flow through over the whole length of the flow passage at substantially the same flowrate (which is calculated corresponding to a minimum flow velocity) ensures that no residual solid material remains left behind or settled out along the length of the flow passage;
By pumping the fluid flow through the flow passage at substantially the same flowrate using a single pumping location for the whole flow passage, means that the present system is less complex than prior art systems because there is no need for secondary pumping in recycle or recirculation side streams and, as a result of using a comparatively lower number of pumps, the present system uses less power and is therefore less expensive to operate;
Using a single pumping location for the flow passage, and arranging this pumping location to be in the flow stream of residual liquid being returned into the flow passage from the step of harvesting the cultivated biological organisms, means that the step of pumping can cause little or no damage to the harvested biological organisms; and
Using a flow passage of serpentine shape minimises the site area, and operating a continuous recycle, closed loop system also means that the risk of water contamination in the flow passage is low when compared with open systems which allow a constantly changing flow composition from external inputs.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose. Terms such as “upper” and “lower”, “above” and “below” and the like are used as words of convenience to provide reference points and are not to be construed as limiting terms.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The preceding description is provided in relation to several embodiments which may share common characteristics and features. It is to be understood that one or more features of any one embodiment may be combinable with one or more features of the other embodiments. In addition, any single feature or combination of features in any of the embodiments may constitute additional embodiments.

In addition, the foregoing describes only some embodiments of the inventions, and alterations, modifications, additions and/or changes can be made thereto without departing from the scope and spirit of the disclosed embodiments, the embodiments being illustrative and not restrictive.

Furthermore, the inventions have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the inventions. Also, the various embodiments described above may be implemented in conjunction with other embodiments, e.g., aspects of one embodiment may be combined with aspects of another embodiment to realise yet other embodiments. Further, each independent feature or component of any given assembly may constitute an additional embodiment.

1. A system for cultivating one or more biological organisms, the system comprising:
a. a flow passage arranged in use for conveying a flow of a fluid containing nutrients and biological organisms suspended in the fluid;
b. a pumping device operable in use to convey the said flow through the flow passage when arranged in fluid communication therewith; and
c. a capture device operable in use to capture a portion of said flow when arranged in fluid communication therewith;
wherein, during operation of the system, the configuration of the flow passage and the operation of the pumping device causes the said flow to be conveyed throughout the flow passage at a substantially uniform flowrate; with
a predetermined depth in the range of more than about 0.2 metres and less than about 0.5 metres; and
a predetermined velocity in the range of more than about 0.3 metres/minute and less than about 1.0 metres/minute,
such that a quantity of the biological organisms which become cultivated in the said flow as a result of exposure to a source of light are captured for removal from the said flow by the capture device.

2. The system as claimed in claim 1, wherein the said flow passage and the pumping device are arranged in use to continuously recirculate the said flow therethrough.

3. The system as claimed in claim 2, wherein the flow passage comprises two flow channels, a distal end region of each flow channel arranged to be in fluid communication with a distal end region of the respective other flow channel.

4-5. (canceled)

6. The system as claimed in claim 2, wherein some or all of said flow passages are adapted to be placed in fluid communication with a respective other flow passage.

7. The system as claimed in claim 6, wherein said adaptation is operable to cause a distal end region of a flow channel of a flow passage to be placed in fluid communication with a respective other distal end region of a flow channel of an adjacent flow passage.

8-11. (canceled)

12. The system as claimed in claim 1, wherein the predetermined depth allows the said light to penetrate substantially all of the way into the said flow to optimise cultivation conditions for the biological organisms.

13-14. (canceled)

15. The system as claimed in claim 1, wherein the predetermined depth is more than about 0.2 metres and less than about 0.4 metres.

16. The system as claimed in claim 1, wherein the predetermined depth corresponds to a quantity of solids in the form of biological organisms of less than about 4% w/v or 40 g/litre to allow said penetration of light.

17. The system as claimed in claim 1, wherein the predetermined velocity of the flow is sufficient to maintain the biological organisms in suspension over the length of the flow passage.

18. (canceled)

19. The system as claimed in claim 1, wherein the predetermined velocity is more than about 0.3 metres/minute and less than about 0.5 metres/minute.

20. The system as claimed in claim 1, wherein the predetermined velocity is more than about 0.3 metres/minute and less than about 0.4 metres/minute.

21. The system as claimed in claim 1, wherein the predetermined velocity is about 0.35 metres/minute.

22. The system as claimed in claim 1, wherein the predetermined velocity corresponds to a quantity of solids in the form of biological organisms of less than about 4% w/v or 40 g/litre which can be maintained in suspension.

23. The system as claimed in claim 1, wherein the capture device for removing a quantity of the cultivated biological organisms from the said flow comprises one or more of the group comprising: vacuum suction device, filtration pump, centrifuge, hydrocyclone, spiral classifier, belt filter, disc filter, filter press, and screening device.

24. The system as claimed in claim 1, wherein after removal of a quantity of the cultivated biological organisms, a flow of residual liquid is returned upstream to the flow passage.

25. The system as claimed in claim 1, wherein the pumping device is a low solids water pump.

26. The system as claimed in claim 25, wherein the pump is arranged to pump the flow of residual liquid upstream to the flow passage after removal of a quantity of the cultivated biological organisms.

27-28. (canceled)

29. A method for cultivating one or more biological organisms, the method comprising the steps of:
a. arranging a pumping device to be in fluid communication with a fluid located in a fluid passage, the fluid containing an amount of nutrients and biological organisms suspended therein;
b. conveying a flow of the said fluid through the flow passage by operation of the pumping device; and
c. capturing a portion of the said flow by operation of a capture device when it is arranged in fluid communication with the said flow;
wherein, the step of operating the pumping device causes the said flow to be conveyed at a substantially uniform flowrate throughout the flow passage, at a predetermined depth in the range of more than about 0.2 metres and less than about 0.5 metres; and at a predetermined velocity in the range of more than about 0.3 metres/minute and less than about 1.0 metres/minute, and a quantity of the biological organisms which become cultivated in the said flow as a result of exposure to a source of light are captured for removal from the said flow by the step of operating the capture device.

30-34. (canceled)

35. The method as claimed in claim 29, further comprising the step of returning a flow of residual liquid upstream to the flow passage after removal of a quantity of the cultivated biological organisms therefrom.

36. (canceled)

37. Apparatus for cultivating one or more biological organisms, the apparatus comprising:
a. a flow passage comprising two or more flow channels arranged in use for conveying a flow of a fluid containing nutrients and biological organisms suspended in the fluid; and
b. a pumping device associated with the flow passage, said pumping device being operable to convey said flow of fluid through the flow passage when arranged in fluid communication therewith at a substantially uniform flowrate throughout the flow passage in a first operating configuration;
the aforementioned apparatus forming a basic unit of a modular system which is arranged in use as a standalone passage for said cultivation in a first, isolated configuration, and wherein, in a second operating configuration, said flow passage of the basic unit is adapted to be placed in fluid communication with the flow passage of at least one further basic unit, and said pumping device is operable to convey said flow of fluid through the flow passage and the further flow passage(s), providing a system with flexibility of residence time for a particular biological organism to develop and to grow while the fluid is moving therethrough.

38. Apparatus as claimed in claim 37, wherein the pumping device is operable to continuously recirculate the said flow through each of the flow passages, if the apparatus is in the second operating configuration.

39. (canceled)

40. Apparatus as claimed in claim 37, wherein said flow passage adaptation is operable to cause a distal end region of a flow channel of a flow passage to be placed in fluid communication with a respective other distal end region of a flow channel of the further flow passage.

41. Apparatus as claimed in claim 37, wherein a capture device is operable in use to capture a portion of said flow when arranged in fluid communication therewith.

42-43. (canceled)

44. A continuous recycle closed loop system for cultivating one or more biological organisms, the system comprising:
a. flow passage arranged in use for conveying a flow of a fluid containing nutrients and biological organisms suspended in the fluid;
b. a low solids water pump pumping device operable in use to convey the said flow through the flow passage when arranged in fluid communication therewith; and
c. a capture device operable in use to capture a portion of said flow when arranged in fluid communication therewith;
wherein, during operation of the system, the configuration of the flow passage and the operation of the low solids water pump causes the said flow to be conveyed at a substantially uniform flowrate throughout the flow passage, and a quantity of the biological organisms which become cultivated in the said flow as a result of exposure to a source of light are captured for removal from the said flow by the capture device, following which the said flow is recirculated using said low solids water pump to a location upstream of the capture device in the flow passage where the biological organisms are being cultivated, without the need for any other pumping arrangement in said flow passage.

45. The system as claimed in claim 44, wherein the source of light for establishing growth conditions is sunlight, and a predetermined depth of the flow passage allows the said light to penetrate substantially all of the way into the said flow to optimise cultivation conditions for the biological organisms.

46. The system as claimed in claim 45, wherein the predetermined depth is more than about 0.2 metres and less than about 0.5 metres.

47. The system as claimed in claim 44, wherein the predetermined depth corresponds to a quantity of solids in the form of biological organisms of less than about 4% w/v or 40 g/litre to allow said penetration of light.

48. The system as claimed in claim 44, wherein the said flow is of a predetermined velocity sufficient to maintain the biological organisms in suspension over the length of the flow passage.

49. The system as claimed in claim 48, wherein the predetermined velocity is in the range of more than about 0.3 metres/minute and less than about 1.0 metres/minute.

50. The system as claimed in claim 44, wherein the predetermined velocity corresponds to a quantity of solids in the form of biological organisms of less than about 4% w/v or 40 g/litre which can be maintained in suspension.

51. A method for cultivating one or more biological organisms in a continuous recycle closed loop, the method comprising the steps of:
a. arranging a low solids water pump to be in fluid communication with a fluid located in a fluid passage, the fluid containing an amount of nutrients and biological organisms suspended therein;
b. conveying a flow of the said fluid through the flow passage by operation of the low solids water pump; and
c. capturing a portion of the said flow by operation of a capture device when it is arranged in fluid communication with the said flow;
wherein, the step of operating the low solids water pump causes the said flow to be conveyed at a substantially uniform flowrate throughout the flow passage, and a quantity of the biological organisms which become cultivated in the said flow as a result of exposure to a source of light are captured for removal from the said flow by the step of operating the capture device, following which the said flow is recirculated using said low solids water pump to a location upstream of the capture device in the flow passage where the biological organisms are being cultivated, without the need for any other pumping arrangement in said flow passage.
